(19) United States
(12) Plant Patent Application Publication
(10) Pub. No.: US 2007/0136918 P1
(43) Pub. Date: Jun. 14, 2007

(54) Vriesea plant named 'Energy'

(75) Inventors: Elly Bak, Rijsenhout (NL); Nico D.M. Steur, Oude Niedorp (NL)

Correspondence Address: FOLEY AND LARDNER LLP;SUITE 500, 3000 K STREET NW, WASHINGTON, DC, 20007, US

(73) Assignee: Corn. Bak B.V.

(21) Appl. No.: 11/298,472

(22) Filed: Dec. 12, 2005

(51) Int. Cl. A01H 5/00

(52) U.S. Cl. PLT/371

(57) ABSTRACT

A new and distinct Vriesea plant named ‘Energy’ characterized as funnel form rosette plant, measuring about 35 cm in height and about 35 cm in diameter and produces compound, yellow, bipinate spike inflorescence, measuring 15 to 20 cm in height, about 2 to 4 branches, measuring about 10 cm to 12 cm in length and 4 cm to 4.5 cm in width, and green foliage, measuring 10 cm to 35 cm in length and about 3.5 cm in width.

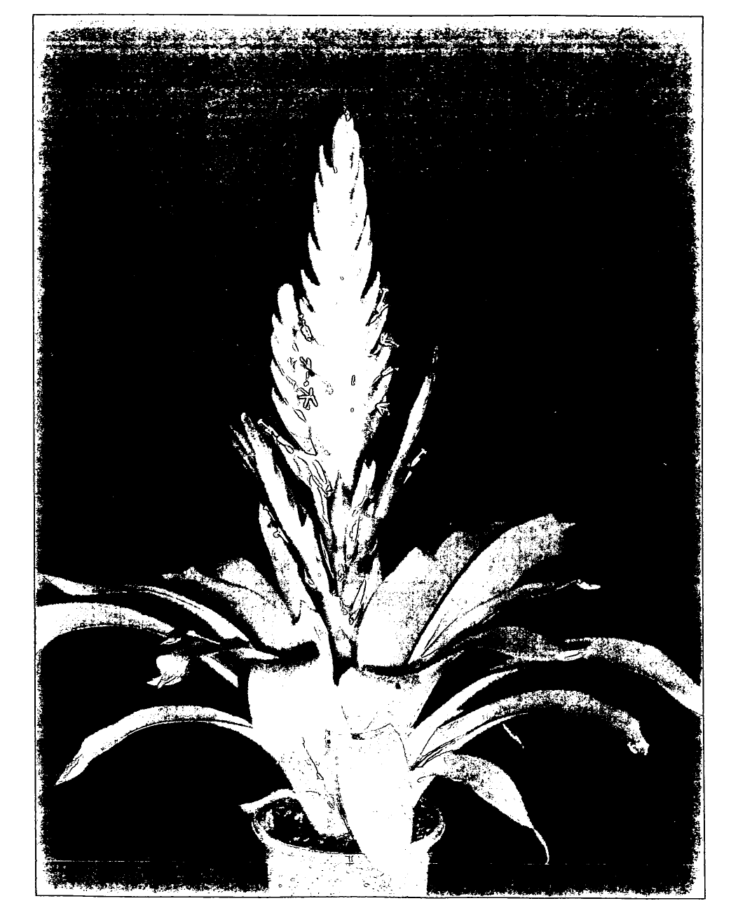

### LATIN NAME OF GENUS AND SPECIES OF THE PLANT CLAIMED

Vriesea sp.

### VARIETY DENOMINATION

‘ENERGY’

### BACKGROUND OF THE INVENTION

The present invention relates to a new and distinct cultivar of Vriesea plant, botanically known as Vriesea sp., hereinafter referred to by the cultivar name ‘Energy’. The genus of Vriesea belongs to the Bromeliaceae family.

The new Vriesea cultivar is a product of a planned breeding program conducted by the inventors, Elly Bak and Nico D. M. Steur, in Assendelft, The Netherlands. The objective of the breeding program is to develop a new Vriesea variety good growing habit and attractive yellow inflorescence.

The new Vriesea cultivar ‘Energy’ originated from a cross-made in a controlled breeding program by the inventors in 1989 in Assendelft, The Netherlands. The female or seed parent is the Vriesea sp. cultivar designated ‘8932807’ (unpatented). The male or pollen parent is the Vriesea sp. cultivar designated ‘8932821’ (unpatented). ‘Energy’ was discovered and selected by the inventors in May of 1992, as a single flowering plant within the progeny of the stated cross in a controlled environment in Assendelft, The Netherlands.

Asexual reproduction of the new cultivar ‘Energy’ performed by vegetative means by tissue culture was first performed in 1997, in Assendelft, The Netherlands. The first ‘Energy’ plants propagated through the use of tissue culture flowered in 2002, in Assendelft, The Netherlands, and have demonstrated that the combination of characteristics as herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be unique characteristics of ‘Energy’ which in combination distinguish this Vriesea as a new and distinct cultivar:
1. Funnel form rosette plant, measuring about 35 cm in height and about 35 cm in diameter;
2. Compound, yellow, bipinate spike inflorescence, measuring 15-20 cm in height;
3. About 2 to 4 branches, measuring about 10 cm to 12 cm in length and 4 cm to 4.5 cm in width; and
4. Green foliage, measuring 10 cm to 35 cm in length and about 3.5 cm in width.

Plants of the parents, ‘8932807’ (unpatented) and ‘8932821’ (unpatented), are no longer available to provide a botanical comparison with the new Vriesea cultivar ‘Energy’. Plants of the new cultivar ‘Energy’ differs from 8932807 with a more branched inflorescence, with less red color on a shorter stem.

Of the many commercial cultivars known to the present inventors, the most similar in comparison to the new Vriesea cultivar ‘Energy’ is the Vriesea cultivar designated ‘Miranda’ (patented, EU 8129). Plants of the new cultivar ‘Energy’ differ from plant of ‘Miranda’ in a more compact growth and a lighter color.

### BRIEF DESCRIPTION OF THE PHOTOGRAPH

The accompanying photograph illustrates the overall appearance of the new Vriesea cultivar ‘Energy’ showing the colors as true as is reasonably possible with colored reproductions of this type. Colors in the photograph may differ slightly from the color values cited in the detailed botanical description which accurately describes the color of ‘Energy’.

The photograph shows a side view perspective of a typical potted flowering plant of ‘Energy’ at 17 months of age.

### DETAILED BOTANICAL DESCRIPTION

‘Energy’ has not been observed under all possible environmental conditions. The phenotype of the new cultivar may vary with variations in environment such as temperature, light intensity, day length, fertilizers and humidity without any change in the genotype of the plant.

The aforementioned photograph, together with the following observations, measurements and values describe the new Vriesea cultivar as grown in a greenhouse in Assendelft, The Netherlands, under conditions which closely approximate those generally used in commercial practice. Plants of ‘Energy’ were grown in a greenhouse with day temperatures ranging from 20° C. to 28° C. and night temperatures ranging from 18° C. to 23° C. No artificial lighting or photoperiodic treatments were conducted, but plants of ‘Energy’ are forced into flowering by adding acetylene. The following fertilizer is added when growing plants of ‘Energy’: 1 part nitrogen, 0.6 parts phosphor, 2 parts Kalium and 0.1 parts magnesium.

Color references are made to The Royal Horticultural Society Colour Chart (RHS), except where general colors of ordinary significance are used. Color values were taken under daylight conditions in a greenhouse in Assendelft, The Netherlands. The age of the plants of ‘Energy’ described is 17 months.
Classification:
   Botanical.—Vriesea sp.
Parentage:
   Male parent.—Vriesea sp. cultivar designated ‘8932821’ (unpatented).
   Female parent.—Vriesea sp. cultivar designated ‘8932807’ (unpatented).
Propagation: Tissue culture
Plant:
   General appearance and form.—Height: About 35 cm Width: About 35 cm Shape: Funnel form rosette.
   Growth habit.—Stemless.
   Plant vigor.—Good.
   Flowering season.—A fully grown plant can flower year round, starting 15-17 weeks after induction of natural light or trough treatment with acetylene.
   Cold tolerance.—Frost tender. Temperatures below 5° C. may damage plants.
   Fragrance.—None.
Foliage:
   Quantity.—About 20 to 30.
   Size of mature leaf.—Length: About 10 to 25 cm Width: About 3.5 cm.
   Overall shape.—Linear, lanceolate.
   Apex shape.—Cuspidate and bending downward.
   Base shape.—Strap-like around central axis.
   Margin.—Entire.
   Texture.—Smooth and glabrous.
   Color.—Leaf color can vary somewhat depending on growing conditions Upper Surface: Mature leaf: Yellow-green, RHS 147 A Immature leaf: Yellow-green, RHS 147 A Lower Surface: Mature leaf: Green, RHS 137 A Immature leaf: Green, RHS 137 A
   Venation.—Pattern: None.
Inflorescence:
   Quantity and form.—1 flower per floral bract, typical for the genus Vriesea; flowering is normally in progression from base to tip of scape Shape: Bipinate spike Size: 15 cm to 20 cm in height.
   Corolla.—Form: Long and narrow, petals and sepals fused at the base around the ovary Number: About 30-80 depending on the size of the plant
   Petals.—Number: 3 per flower Length: About 4 cm Width: About 0.6 cm Overall Shape: Ligulate Apex Shape: Cuspidate Base Shape: Fused Color: Upper surface: Yellow, RHS 5A Lower surface: Yellowish translucent, closest to RHS 5A.
   Sepals.—Number: 3 per flower Length: About 3 cm Width: About 1.1 cm Overall Shape: Ligulate Apex Shape: Acute Base Shape: Fused Color: Translucent, closest to RHS 5A.
   Branches.—Quantity: About 2 to 4 Length: About 10 cm to 12 cm Width: About 4 cm to 4.5 cm Texture: Spike Color: Red, RHS 42A, and yellow, RHS 13A.
   Bracts.—Quantity of bracts on main spike: About 12 to 18 Arrangement: Alternate Size: Length: About 4.0 cm to 4.5 cm Width: About 4.0 cm to 6.0 cm Overall shape: Ovate Color: Yellow, RHS 13A, with red, RHS 42A, at base.
Reproductive organs:
Androecium:
   Stamen.—Number: 6 per flower Length: About 5 cm Diameter: About 0.1 cm Color: Cream, closest to RHS 11 D.
   Anther.—Shape: Versatile Length: About 0.7 cm Color: Yellow, closest to RHS 11 C.
   Pollen.—Amount: Scarce Color: Yellow, closest to RHS 11 A.
Gynoecium:
   Pistil.—Number: 1 per flower Length: About 5 cm.
   Stigma.—Shape: 3-parted Width: About 0.3 cm Color: Yellow-green, closest to RHS 150 C.
   Style.—Length: About 5 cm Color: Yellow, closest to RHS 11 C.
   Ovary.—Shape: Conical Length: About 0.5 cm Diameter: About 0.2 cm Color: Cream, closest to RHS 11D.
Seeds: none produced
Fruit: None produced
Disease/pest resistance: No information is currently available.
Disease/pest susceptibility: No information is currently available.

I claim:

1. A new and distinct Vriesea plant named ‘Energy’, substantially as illustrated and described herein.
